(19) United States
(12) Patent Application Publication
(10) Pub. No.: US 2025/0025647 A1
(43) Pub. Date: Jan. 23, 2025

(54) REACTIVATION OF AND RESTORATION OF ELECTRICAL SIGNALING BY NEURONS INVOLVED IN CONTROLLING BRAIN FUNCTION

(71) Applicant: Anecare, LLC, Salt Lake City, UT (US)

(72) Inventor: Michael Slatter, Park City, UT (US)

(21) Appl. No.: 18/223,426

(22) Filed: Jul. 18, 2023

(51) Int. Cl. A61M 16/00; A61M 16/01

(52) U.S. Cl. CPC A61M 16/0045; A61M 16/0093; A61M 16/01; A61M 2202/0208

(57) ABSTRACT

A system for reversing the effects of inhaled anesthesia reactivates and restores electrical signaling of neurons that control brain function of a subject. The system is used to administer increased inhaled carbon dioxide to a subject while causing an increase in the subject's respiratory rate and tidal volume and, thus, an increase in the subject's minute ventilation. The changes in the subject's respiration are tailored to increase extracellular acidification around neurons that control brain function, which have been affected by the inhaled anesthesia, and to inhibit ion channel activity (e.g., TREK-1 ion channel activity, etc.) to reactivate and restore electrical signaling by such neurons.

### TECHNICAL FIELD

This disclosure relates generally to the reactivation of and restoration of electrical signaling by neurons that control brain function (e.g., consciousness, respiration (or breathing), etc.) of the subject that have been inactivated by the administration of inhaled anesthesia to a subject and, more specifically, to increasing inhaled carbon dioxide along with increasing respiratory rate and tidal volume, or to increasing minute ventilation, in a manner tailored to inhibit ion channel activity (e.g., TREK-1 ion channel activity, etc.) to reactivate and restore electrical signaling by such neurons.

### RELATED ART

Inhaled anesthesia, or the use of volatile anesthetics, are commonly used to sedate subjects during surgery. Inhaled anesthesia is also used to sedate subjects for other purposes and to treat certain conditions.

While the use of inhaled anesthesia is typically quite effective, the duration of time required to reverse the effects of inhaled anesthesia is often undesirably lengthy. Prolonged exposure to anesthesia may result in nausea and vomiting and/or contribute to airway complications, such as in patients with obstructive sleep apnea. In addition, inhaled anesthesia can have long-term effects on some subjects, such as postoperative cognitive decline or dysfunction (POCD) and the concomitant reduction in quality of life in the elderly. More specifically, delirium is the most common surgical complication in people who are at least 65 years old. It has been estimated that 20% to 30% of people 65 and older who experience post-surgical delirium also experience cognitive decline or dysfunction. It has also been estimated that, in the United States alone, post operative delirium (POD) results in nearly $33 billion in health care costs each year.

Techniques for accelerating the rate of reversal of the effects of inhaled anesthesia, or volatile anesthetics, on a subject have included increasing the rate at which blood flows through the subject's brain. See, e.g., U.S. Pat. No. 7,353,825. The rate at which blood flows through the subject's brain may be increased by elevating the level (i.e, partial pressure) of carbon dioxide, or CO₂, in the subject's blood. See, e.g., id. One way of elevating the level of carbon dioxide within the subject's blood is to cause the subject to rebreathe exhaled gases. See, e.g., id. Exhaled anesthesia is, of course, removed from the exhaled gases to prevent its re-inhalation, which would impede anesthesia reversal. See, e.g., id. The increased carbon dioxide levels cause increased cerebral blood flow and an increase in breathing rate and an increase in tidal volume, which may increase the rate at which the lungs remove inhaled volatile anesthetics from the blood and transport these anesthetics out of the subject's body. See, e.g., id.

While increasing the amount of carbon dioxide in gases inhaled by the subject elevates respiratory rate and tidal volume, the addition of carbon dioxide could also be detrimental, even harmful, as high levels, or amounts (i.e., partial pressures), of carbon dioxide in gases inhaled by the subject corresponds to high levels of acidity within the cells.

While existing techniques are useful for discontinuing the delivery of inhaled anesthesia in many subjects, the effectiveness of this process can be limited, with POD and POCD still occurring in an undesirably large number of subjects.

### SUMMARY

In various aspects, methods, apparatuses, and systems for reactivating and restoring electrical signaling by neurons that control brain functions (e.g., consciousness, respiration, etc.) and that have been inactivated by inhaled anesthesia, or one or more volatile anesthetics. Such methods, apparatuses, and systems may accelerate reversal of the effects of inhaled anesthesia, in a subject. The methods, apparatuses, and systems of this disclosure may be employed once administration of the inhaled anesthesia to the subject is complete. Methods, apparatuses, and systems according to this disclosure may effectively wash anesthetics from the central nervous system (CNS).

A method for reactivating and restoring electrical signaling by neurons that control brain function following the administration of inhaled anesthesia may include causing the subject to breathe an above-ambient amount of carbon dioxide while increasing the respiratory rate and tidal volume of the subject, or increasing minute ventilation (respiratory rate×tidal volume) of the subject. The subject's minute ventilation may be increased in a controlled manner. Such a technique may also be referred to as “hypercapnic hyperpnoea.”

An above-ambient amount of carbon dioxide may be provided to the subject in any of a variety of suitable ways. For example, exhaled gases may be provided to the subject; i.e., the subject may rebreathe exhaled carbon dioxide. As another example, the subject may be caused to breathe an elevated level of carbon dioxide by supplementing the subject's respiratory gases (i.e., the gases inhaled by the subject) with carbon dioxide. The amount of carbon dioxide in the subject's respiratory gases may be controlled in a manner that controls the minute ventilation of the subject.

As an alternative or in addition to controlling minute ventilation by controlling the amount (i.e., partial pressure) of carbon dioxide in the subject's respiratory gases, the minute ventilation may be controlled by mechanically controlling the respiratory rate and tidal volume of the subject's respiration.

The minute ventilation of the subject may be increased so that it exceeds normal minute ventilation for the subject (e.g., a normal anesthetized minute ventilation for the subject, an average anesthetized minute ventilation for the subject, etc.). The respiratory rate of the subject may be increased so that it exceeds a normal respiratory rate for the subject (e.g., a normal anesthetized respiratory rate for the subject, an average anesthetized respiratory rate for the subject, etc.). The tidal volume of the subject may be increased so that it exceeds a normal tidal volume for the subject (e.g., a normal anesthetized tidal volume for the subject, an average anesthetized tidal volume for the subject, etc.) but not so much that it dilutes the carbon dioxide in the subject's respiratory gases to ineffective levels that will not allow anesthesia to be cleared at a desired rate, or within a desired duration for a particular type of inhaled anesthesia. Increasing the tidal volume of respiration by the subject may control (e.g., decrease) respiratory acidosis and, thus, may counteract the potentially detrimental effects of having the subject inhale elevated levels of carbon dioxide.

The extent to which the minute ventilation of the subject, or the respiratory rate and tidal volume of the subject, may be increased may be tailored to inhibit ion channel activity in and to restore electrical signaling by neurons involved in controlling brain function of the subject, such as the subject's consciousness and/or respiration. More specifically, the controlled increase in minute ventilation, or the increase in respiratory rate and tidal volume, of the subject may be tailored to decrease respiratory acidosis and, thus, decrease intracellular acidification of neurons involved in controlling brain function of the subject, and/or increase acidification around, or outside of, the neurons involved in controlling brain function of the subject (i.e., cause extracellular acidification relative to these neurons). Even more specifically, the controlled increase in minute ventilation, or the controlled increase in respiratory rate and tidal volume, combined with increased carbon dioxide of the subject may be tailored to increase extracellular acidification to an extent that will inhibit TREK-1 ion channel activity, thereby shutting down the passage of potassium ions out of neurons involved in controlling brain function (e.g., consciousness, respiration, etc.) of the subject through their cell membranes, or potassium production by the cell membranes of the neurons involved in controlling brain function of the subject. Inhibition of TREK-1 ion channel activity in the cell membrane of these neurons may restore electrical signaling by the neurons and, thus, restore brain function (e.g., consciousness, respiration, etc.) controlled by the neurons.

In addition to causing the subject to breathe an above-ambient amount of carbon dioxide in a controlled manner and increasing the minute ventilation of the subject in a controlled manner, a method for reversing the effects of inhaled anesthesia of this disclosure may include filtering anesthesia from gases exhaled and/or reinhaled, or rebreathed, by the subject.

Another aspect of this disclosure is a system that reactivates and restores electrical signaling by neurons that control brain function of a subject once administration of inhaled anesthesia to the subject is complete. Such a system may include a ventilator, a breathing circuit, and an anesthesia reversal device.

The ventilator may ventilate the subject at a minute ventilation that exceeds a normal minute ventilation for the subject (e.g., a normal anesthetized minute ventilation for the subject, an average anesthetized minute ventilation for the subject, etc.) while maintaining an optimum amount of carbon dioxide in the subject's respiratory gases, thereby maintaining optimum levels of carbon dioxide in the subject's blood (i.e., partial pressure of carbon dioxide) and neurons involved in controlling brain function. Accordingly the ventilator may increase one or both of a respiratory rate and a tidal volume of the subject in a controlled manner. The ventilator may increase the respiratory rate of the subject to a respiratory rate that exceeds the normal respiratory rate for the subject (e.g., the normal anesthetized respiratory rate for the subject, the average anesthetized respiratory rate for the subject, etc.). The ventilator may increase the tidal volume of the subject to a tidal volume that exceeds the normal tidal volume for the subject (e.g., the normal anesthetized tidal volume for the subject, the average anesthetized tidal volume for the subject, etc.). The extent to which the ventilator increases the respiratory rate and the tidal volume and, thus, the minute ventilation of the subject may be limited to prevent dilution of the carbon dioxide in the subject's respiratory gases to ineffective levels that will not allow anesthesia to be cleared at a desired rate, or within a desired duration for a particular type of inhaled anesthesia.

The breathing circuit may establish communication between the ventilator and the subject. The breathing circuit may comprise any breathing circuit suitable for use with subjects who receive inhaled anesthesia. The breathing circuit may include an inspiratory limb and an expiratory limb. In addition, the breathing circuit may include an anesthesia filter, which may be associated with either the inspiratory limb or the expiratory limb.

The anesthesia reversal device may communicate with the breathing circuit in a manner that causes the subject to inhale an above-ambient amount of carbon dioxide. For example, the anesthesia reversal device may cause the subject to rebreathe exhaled carbon dioxide. As another example, the anesthesia reversal device may add supplemental carbon dioxide to gases that are to be inhaled by the subject.

In some embodiments, a system that reactivates and restores electrical signaling by neurons that control brain function of a subject who has received inhaled anesthesia may also include an oxygen source, which may introduce supplemental oxygen into gases inhaled by the subject.

The system may be programmed or otherwise tailored such that its ventilator and anesthesia reversal device may cause the subject to breathe in a manner that inhibits ion channel activity in and restores electrical signaling by neurons involved in controlling brain function, such as the subject's consciousness and/or respiration. More specifically, the system may be programmed to increase the minute ventilation, or to increase the respiratory rate and tidal volume and increase carbon dioxide, of the subject in a manner that increases acidification around the neurons involved in controlling brain function of the subject, or causes extracellular acidification of these neurons. Even more specifically, the system may be programmed to increase the minute ventilation, or to increase the respiratory rate and tidal volume, of the subject in a manner that increases extracellular acidification to an extent that will inhibit TREK-1 ion channel activity in the neurons involved in controlling brain function (e.g., consciousness, respiration, etc.) of the subject. Inhibition of TREK-1 ion channel activity in these neurons following administration of anesthesia may restore electrical signaling by the neurons and, thus, restore brain function (e.g., consciousness, respiration, etc.) controlled by the neurons.

By restoring electrical signaling in neurons involved in controlling brain function, such as the subject's consciousness and/or respiration, methods and systems according to this disclosure may reduce the incidence of post-surgical complications attributable to prolonged exposure to anesthesia. The complications that are reduced may include, but are not limited to, nausea, vomiting, airway complications (e.g., due to obstructive sleep apnea, etc.), post-surgical delirium, and/or cognitive decline or dysfunction. Thus, the methods, apparatuses, and systems of this disclosure may prevent, or reduce the incidence of, post-surgical delirium, including post-surgical delirium in people who are at least 65 years old, who are also referred to herein as “seniors.”

By reducing post-surgical complications and the potential long-term effects of post-surgical complications, the use of methods, apparatuses, and systems of this disclosure may reduce healthcare costs.

Other aspects of the disclosed subject matter, as well as features and advantages of various aspects of the disclosed subject matter, should become apparent to those of ordinary skill in the art through consideration of the ensuing description, the accompanying drawings, and the appended claims.

### BRIEF DESCRIPTION OF THE DRAWINGS

In the drawings:

FIG. 1 provides a schematic representation of a neuronal axon potential, which is the electrical signal a neuron sends from its cell body and along an axon of the neuron;

FIGS. 2 and 3 respectively depict the depolarization and repolarization that occur at various locations along the length of the axon of the neuron to transmit the signal along the length of the axon as a signal is conveyed along the length of the axon;

FIG. 4 is a schematic representation of a cell membrane of the axon of the neuron, showing how ions move into and out of the axon to change the potential across the cell membrane;

FIG. 5 is a graph showing changes in the potential across the cell membrane as depolarization and repolarization occur at a location along the length of the axon to transmit the signal (i.e., a positive potential) and to reset the location to a resting potential (i.e., a negative potential); and

FIG. 6 is a schematic representation of a system of this disclosure, which includes a ventilator, a breathing circuit, and an anesthesia reversal device configured to reactivate and restore electrical signaling by neurons that control brain functions (e.g., consciousness, respiration, etc.) and that have been inactivated by inhaled anesthesia.

### DETAILED DESCRIPTION

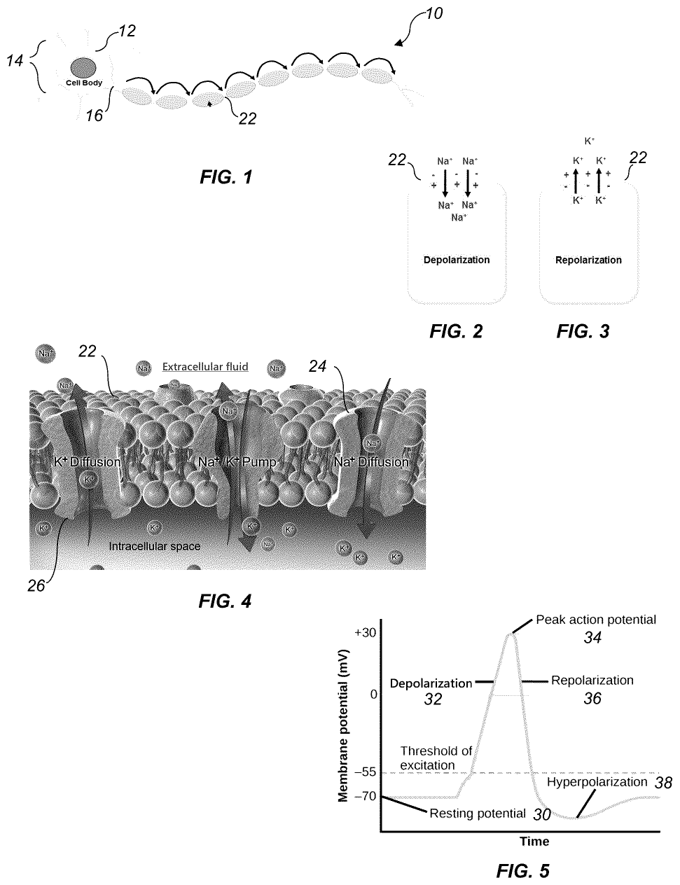

FIGS. 1-5 provide a representation of the manner in which neurons transmit impulses. The effects of inhaled anesthesia, or volatile anesthetics, on neurons that control brain function, such as consciousness and/or respiration, will also be described in reference to FIGS. 1-5.

FIG. 1 illustrates a neuron 10, which includes a cell body 12, dendrites 14 that receive signals, and an axon 16 that transmits signals. The axon 16 is an elongated member of the neuron 10.

The neuron 10 sends a signal from its cell body 12, down its axon 16 by way of a neuronal action potential. A neuronal action potential occurs as the potential across the cell membrane 22 rapidly rises and falls. More specifically, the neuronal action potential is created by depolarizing the cell membrane 22. A signal is transmitted along the length of the axon 16 as the cell membrane 22 along the length of the axon 16 is depolarized. The potential across the cell membrane 22 is then reset by repolarizing the cell membrane 22.

Depolarization and repolarization occur as ions pass through and are transported through the cell membrane 22. FIGS. 2 and 3 respectively depict depolarization and repolarization of the cell membrane 22.

As shown in FIG. 2, during depolarization, sodium ions Na⁺ from outside the cell travel through the cell membrane 22 into the neuron 10. More specifically, as shown in FIG. 4, sodium ions Na⁺ enter the neuron 10 through open voltage-gated sodium ion channels 24 in the cell membrane 22. With added reference to FIG. 5, as the sodium ions Na⁺ enter the cell membrane 22, the neuron 10 depolarizes 32, increasing the potential across the cell membrane 22 from a resting potential 30 of −70 millivolts (mV) to a peak action potential 34 of +30 mV.

After the cell membrane 22 has depolarized, it typically repolarizes. During repolarization, potassium ions K⁺ travel out of the neuron 10 through the cell membrane 22, as shown in FIG. 3. More specifically, the sodium ion channels 24 shown in FIG. 4 close and potassium ion channels 26 in the cell membrane 22, also shown in FIG. 4, open, causing potassium ions K⁺ to exit the neuron 10. With added reference to FIG. 5, as the neuron 10 repolarizes 36, the membrane potential decreases from the peak action potential 34 of +30 mV. Under normal conditions, the neuron 10 momentarily hyperpolarizes 38 before the potassium ion channels 26 close, to a potential of less than −70 mV and the neuron 10 then returns to its resting potential 30, enabling it to transmit another signal.

Inhaled anesthesia causes neurons 10 that are involved in controlling brain functions, such as consciousness and/or respiration, to enter into prolonged hyperpolarized states, preventing the neuron 10 from resetting and thereby shutting down the electrical signaling. Inhaled anesthesia is believed to function by disrupting so-called “lipid rafts,” which are cholesterol-rich and sphingolipid-rich (e.g., sphingomyelin-rich, etc.) areas of a cell membrane 22 of a neuron 10. More specifically, inhaled anesthesia is believed to function by disrupting GM1 lipid rafts (which include the sphingolipid monosialotetrahexosylganglioside1 (GM1) in the cell membrane 22 of neurons 10 that control brain functions, such as consciousness and respiration. When inhaled anesthesia disrupts GM1 lipid rafts, the GM1 lipid rafts release the enzyme Phospholipase D2 (PLD2). The released PLD2 gathers at less preferred Phosphatidylinositol 4,5-bisphosphate (PIP2) lipid clusters, where potassium ion channels 26 known as TREK-1 ion channels 26 are located. The PLD2 then activates, or opens, the TREK-1 ion channels 26, which continuously causes potassium ions K⁺ to flow out of the cell membrane 22 and, thus, places the neuron 10 into a prolonged hyperpolarized state, preventing the neuron 10 from resetting and, thus, impeding the ability of the neuron 10 and its axon 16 to transmit electrical signals, thereby causing loss of consciousness and sedation.

As inhaled anesthesia clears, PLD2 may dissociate from the TREK-1 ion channels 26, enabling the TREK-1 ion channels 26 to close, which may enable the neuron 10 to resume its cycle of depolarization, repolarization, and hyperpolarization and, thus, enable signal sending by the neuron 10 to resume.

If the amount of carbon dioxide within respiratory gases is too high as inhaled anesthesia dissipates, respiratory acidosis may occur. Respiratory acidosis causes the blood to become acidic, which, in turn, decreases the intracellular pH, or increases the intracellular acidity, within neurons 10 that control brain functions, such as consciousness and respiration. A decrease in the intracellular pH, or increases in the intracellular acidity, within neurons 10 that control brain functions activates the TREK-1 ion channel 26 causing the neuron 10 to exude potassium ions K⁺ through the cell membrane 22, which inhibits the return of the neuron 10 to its resting potential and shuts down signaling of the neuron 10 and may effectively prolong the effects of the inhaled anesthesia and prolong or prevent the subject's recovery from the inhaled anesthesia.

Anesthesia reversal according to this disclosure is tailored to accelerate the removal and to reverse the effects of inhaled anesthesia in neurons 10 that control brain functions, such as consciousness and/or respiration, while avoiding, or without causing, respiratory acidosis. The controlled increase in respiratory rate and tidal volume, combined with increased carbon dioxide of the subject may be tailored to decrease respiratory acidosis and increase extracellular acidification to an extent that will inhibit TREK-1 ion channel 26 activity, thereby shutting down the passage of potassium ions out of the neurons 10 through their cell membranes 22, or potassium production by the cell membranes 22 of the neurons 10, that have been activated by inhaled anesthesia to restore electrical signaling in neurons 10 that control brain functions, such as consciousness and/or respiration. Thus, the process of anesthesia reversal avoids respiratory acidosis and may also reduce post-surgical complications, such as delirium and cognitive decline or dysfunction, that may result from post-surgical delirium.

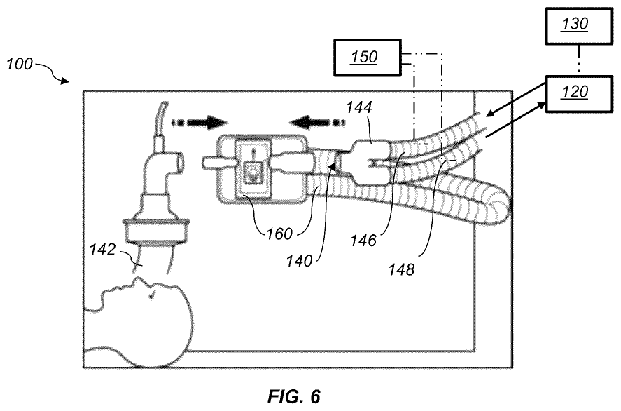

With reference to FIG. 6, an embodiment of a system 100 is depicted that facilitates the emergence of a subject from inhaled anesthesia in a manner that removes inhaled anesthesia and restores electrical signaling of neurons that control brain functions (e.g., consciousness, respiration, etc.) and that have been inactivated by inhaled anesthesia. Such a system 100 may be referred to as an “anesthesia reversal system.” Such a system 100 may minimize the likelihood of post-surgical complications, such as delirium and cognitive decline or dysfunction, that may result from post-surgical delirium. The system 100 includes a ventilator 120, a breathing circuit 140, and an anesthesia reversal device 160. Optionally, the anesthesia reversal system 100 may include an anesthesia filter 150 associated with the breathing circuit 140 and/or an oxygen source 130 associated with the ventilator 120.

The ventilator 120 may comprise any suitable ventilator (e.g., a manually operated ventilator (or bag), a mechanical ventilator, etc.) that can ventilate a subject at a controlled respiratory rate and tidal volume to provide a controlled minute ventilation. When the subject is anesthetized, the ventilator 120 may ventilate the subject at an anesthetized respiratory rate and at an anesthetized tidal volume to provide an anesthetized minute ventilation. As the subject recovers from the inhaled anesthesia (e.g., post-surgically, etc.), the ventilator 120 may ventilate the subject at a recovery respiratory rate that exceeds the subject's anesthetized respiratory rate and tidal volume that exceeds the subject's anesthetized tidal volume to provide a recovery minute ventilation that exceeds the subject's anesthetized minute volume. The recovery respiratory rate may be at least 10 breaths per minute (e.g., 10 breaths per minute to 12 breaths per minute, up to 20 breaths per minute, 16-20 breaths per minute, etc.). The recovery tidal volume may be at least 8 mL/kg of body weight of the subject (e.g., 8 mL/kg to 10 mL/kg of body weight of the subject, etc.). The ventilator 120 may provide or assist in providing (i.e., along with spontaneous breaths) the subject with a recovery minute ventilation of at least 80 mL/kg/min (e.g., a recovery minute ventilation of 80 mL/kg/min to 120 mL/kg/min, etc.).

In embodiments where the system includes an oxygen source 130, any suitable type of oxygen source 130 may be employed. The oxygen source 130 may deliver at least 10 L of oxygen to the ventilator 120 and, thus, to the subject each minute.

In some embodiments, the ventilator 120 may be programmed to provide a particular anesthetized respiratory rate and anesthetized tidal volume to provide a particular anesthetized minute ventilation. Such a ventilator 120 may also be programmed to provide a particular recovery respiratory rate and recovery tidal volume to provide a particular recovery minute ventilation. In addition, such a ventilator 120 may be programmed to deliver a controlled amount of oxygen to the subject and, thus, to provide a controlled rate of oxygen delivery while the subject inhales anesthesia and as the subject recovers from the inhaled anesthesia.

The ventilator 120 may allow for and account for spontaneous ventilation by the subject. The ventilator 120 may be programmed to provide the subject with a recovery respiratory rate and a recovery tidal volume to provide the subject with a target minute volume.

The breathing circuit 140 establishes communication between the ventilator 120 and the subject. The breathing circuit 140 may include an endotracheal tube or supraglottic airway device 142, a Y-piece 144, an inspiratory limb 146, and an expiratory limb 148. The endotracheal tube or supraglottic airway device 142 may interface with the subject (e.g., it may be inserted into the subject's airway). The Y-piece 144 of the breathing circuit 140 connects the endotracheal tube or supraglottic airway device 142 to the inspiratory limb 146 and the expiratory limb 148. The breathing circuit 140 may comprise any suitable breathing circuit used in the delivery of inhaled anesthesia to the subject. Without limitation, the breathing circuit 140 may comprise a so-called circle breathing circuit.

In embodiments where the anesthesia reversal system 100 includes an anesthesia filter 150 associated with the breathing circuit 140, anesthesia filter 150 may be associated with the inspiratory limb 146 of the breathing circuit 140 and/or with the expiratory limb 146 of the breathing circuit 140. The breathing circuit 140 may be configured in such a way as to provide selectivity over whether gases exhaled by the subject flow through the anesthesia filter 150 (i.e., selectivity over whether the exhaled gases bypass or are directed through the anesthesia filter 150). For example, while the subject continues to receive inhaled anesthesia (e.g., during a surgical procedure, etc.), gases that have been exhaled by the subject may bypass the anesthesia filter 150. Following the administration of inhaled anesthesia to the subject (e.g., after the surgical procedure is complete, etc.), gases that have been exhaled by the subject may be directed through the anesthesia filter 150.

The anesthesia reversal device 160 is associated with the breathing circuit 140 in such a way that the anesthesia reversal device 160 can affect the amount of carbon dioxide in gases inhaled by the subject. When employed, the anesthesia reversal device 160 may facilitate the delivery of an above ambient amount of carbon dioxide to the subject. Without limitation, the anesthesia reversal device 160 may comprise an anesthesia reversal device that may cause a subject to rebreathe exhaled gases, including exhaled carbon dioxide, following administration of inhaled anesthesia. The ANEclear® anesthesia reversal device available from Anecare, LLC of Salt Lake City, Utah is an example of such an anesthesia reversal device. The anesthesia reversal device 160 may be positioned at a suitable position along the breathing circuit 140, for example, between the endotracheal tube 142 of the breathing circuit 140 and the Y-piece 144 between the inspiratory limb 146 and the expiratory limb 148 of the breathing circuit 140.

The ventilator 120 and the anesthesia reversal device 160 of the system 100 may cause the subject to breathe in a manner that inhibits ion channel activity in and restores electrical signaling by neurons 10 (FIGS. 1-5) involved in controlling brain functions, such as consciousness and/or respiration of the subject. More specifically, the ventilator 120 and the anesthesia reversal device 160 can cause the subject to breathe in a manner that achieves extracellular acidification and inhibits TREK-1 ion channel activity in and restores electrical signaling by neurons 10 involved in controlling brain functions, such as consciousness and respiration.

An embodiment of a method of this disclosure facilitates the emergence of a subject from inhaled anesthesia in a manner that reactivates and restores electrical signaling of neurons that control brain functions (e.g., consciousness, respiration, etc.) and that have been inactivated by inhaled anesthesia. The use of such a method may minimize the likelihood of post-surgical complications, such as delirium and cognitive decline or dysfunction, that may result from post-surgical delirium. The method may include causing a subject to breathe an above-ambient amount of carbon dioxide and increasing the respiratory rate and a tidal volume of respiration of the subject. The above-ambient carbon dioxide and increased respiratory rate and tidal volume may be tailored to inhibit TREK-1 ion channel activity in neurons involved in controlling brain functions (e.g., consciousness, respiration, etc.) of the subject.

Causing the subject to breathe the above-ambient amount of carbon dioxide may comprise administering the above-ambient amount of carbon dioxide to the subject (e.g., by manually ventilating the subject, by mechanically ventilating the subject, etc.). The carbon dioxide may be supplied from an external source, from rebreathing exhaled gases, or from a combination an external source and rebreathing. The amount of carbon dioxide inhaled by the subject may be controlled. In embodiments where exhaled gases are rebreathed, the method may include filtering anesthesia from the exhaled gases.

In the method, the respiratory rate of the subject may be increased from an anesthetized respiratory rate or even from a normal respiratory rate for the subject to a recovery respiratory rate. As an example, the recovery respiratory rate may be increased to at least 10 breaths per minute (e.g., to a range of 10 breaths per minute to 12 breaths per minute, to up to 20 breaths per minute, 16-20 breaths per minute, etc.).

The tidal volume of the subject's respiration may be increased from an anesthetized tidal volume or even from a normal tidal volume of the subject to a recovery tidal volume. As an example, the recovery tidal volume may be increased to at least 8 mL/kg of body weight of the subject. As another example, the recovery tidal volume may be increased to 8 mL/kg to 10 mL/kg of body weight of the subject.

The subject's minute ventilation may be increased from an anesthetized minute ventilation to a recovery minute ventilation. As an example, the method may increase the subject's minute ventilation to at least 80 mL/kg/min (e.g., a recovery minute ventilation of 80 mL/kg/min to 120 mL/kg/min, etc.).

Such a method may provide a change the pH in the subject's brain that is tailored to promote extracellular acidification and, thus, to activate and restore electrical signaling by neurons that control brain functions (e.g., consciousness, respiration, etc.) and that have been inactivated by inhaled anesthesia.

In a specific embodiment of a method according to this disclosure, subjects were ventilated at a recovery respiratory rate of 16-20 breaths per minute at tidal volumes of 8 mL/kg of body weight to 10 mL/kg of body weight. On average, the method shortened the time-to-consciousness for subjects who were anesthetized with desflurane by 5 minutes and shortened the time-to-consciousness for subjects who were anesthetized with sevoflurane by 7 minutes and shortened the time-to-consciousness for subjects who were anesthetized with isoflurane by 11 minutes, resulting in about a reduction of at least 50% (actually, about 60%) in the amount of time it took the subjects to emerge from the inhaled anesthesia. The accelerated emergence from inhaled anesthesia decreased the amount of time subjects spent in the recovery unit, on average, by about 23 minutes, or 25%.

A method according to this disclosure may be used with subjects who are expected to be at risk for post-surgical complications, such as delirium, and, thus, cognitive decline or dysfunction that may result from post-surgical delirium. Thus, a method of this disclosure may include predicting whether a subject will benefit from use of the disclosed methods, apparatuses, and/or systems. Such a prediction may be based upon a variety of factors, including, without limitation, the subject's age, medical condition, or the like.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A method for accelerating reversal of the effects of inhaled anesthesia on a subject once administration of the inhaled anesthesia to the subject is complete, comprising:
causing the subject to breathe an above-ambient amount of carbon dioxide;
increasing a respiratory rate by the subject to above a normal anesthetized respiratory rate for the subject; and
increasing a tidal volume of respiration by the subject to above a normal anesthetized tidal volume for the subject,
the causing the subject to breathe the above-ambient amount of carbon dioxide, the increasing the respiratory rate, and the increasing the tidal volume together tailored to inhibit TREK-1 ion channel activity in and to restore electrical signaling by neurons involved in controlling brain function including consciousness and respiration of the subject.

2. The method of claim 1, wherein causing the subject to breathe an elevated level of carbon dioxide includes causing the subject to rebreathe exhaled carbon dioxide.

3. The method of claim 2, further comprising:
filtering anesthesia from gases exhaled and/or reinhaled by the subject.

4. The method of claim 1, wherein increasing the respiratory rate comprises increasing the respiratory rate to at least 10 breaths per minute.

5. The method of claim 4, wherein increasing the respiratory rate comprises increasing the respiratory rate to 10 breaths per minute to 12 breaths per minute.

6. The method of claim 1, wherein increasing the tidal volume comprises increasing the tidal volume to at least 8 mL/kg of body weight of the subject.

7. The method of claim 6, wherein increasing the tidal volume comprises increasing the tidal volume to 8 mL/kg to 10 mL/kg of body weight of the subject.

8. The method of claim 1, further comprising:
filtering anesthesia from gases exhaled and/or reinhaled by the subject.

9. The method of claim 1, comprising:
causing the subject to breathe at least 10 L of oxygen per minute.

10. The method of claim 1, wherein the causing the subject to breathe the above-ambient amount of carbon dioxide, the increasing the respiratory rate, and the increasing the tidal volume are together tailored to cause extracellular acidification to inhibit the TREK-1 ion channel activity in and to restore the electrical signaling by the neurons involved in controlling brain function.

11. A method for accelerating reversal of the effects of inhaled anesthesia on a subject once administration of the inhaled anesthesia to the subject is complete, comprising:
causing the subject to rebreathe exhaled carbon dioxide;
increasing a respiratory rate by the subject to at least 10 breaths per minute; and
increasing a tidal volume of respiration by the subject to at least 8 mL/kg of body weight of the subject.

12. The method of claim 10, further comprising:
filtering anesthesia from gases exhaled and/or reinhaled by the subject.

13. The method of claim 11, wherein increasing the tidal volume of respiration by the subject controls respiratory acidosis while causing the subject to rebreathe exhaled carbon dioxide.

14. A system for accelerating reversal of the effects of inhaled anesthesia on a subject once administration of the inhaled anesthesia to the subject is complete, comprising:
a ventilator that ventilates the subject at a respiratory rate that exceeds a normal anesthetized respiratory rate for the subject and at a tidal volume that exceeds a normal anesthetized tidal volume for the subject;
a breathing circuit that establishes communication between the ventilator and the subject; and
an anesthesia reversal device in communication with the breathing circuit in a manner that causes the subject to inhale an above-ambient amount of carbon dioxide,
with the ventilator and the anesthesia reversal device causing the subject to breathe in a manner that inhibits ion channel activity in and restores electrical signaling by neurons involved in controlling brain function including consciousness and respiration of the subject.

15. The system of claim 14, wherein the anesthesia reversal device causes the subject to rebreathe exhaled carbon dioxide.

16. The system of claim 15, further comprising:
an anesthesia filter associated with an inspiratory limb of the breathing circuit and/or an expiratory limb of the breathing circuit.

17. The system of claim 14, wherein the ventilator ventilates the subject at a minute ventilation of at least 80 mL/kg of body weight of the subject.

18. The system of claim 17, wherein the ventilator ventilates the subject at a minute ventilation of 80 ml/kg to 120 ml/kg of body weight of the subject.

19. The system of claim 14, further comprising:
an oxygen source that enables the ventilator to deliver at least 10 L of oxygen to the subject per minute.

20. The system of claim 14, wherein the ventilator and the anesthesia reversal device can cause the subject to breathe in a manner that achieves extracellular acidification and inhibits TREK-1 ion channel activity in and restores electrical signaling by neurons involved in controlling brain function including consciousness and respiration of the subject.
